(19) United States
(12) Patent Application Publication
(10) Pub. No.: US 2015/0355413 A1
(43) Pub. Date: Dec. 10, 2015

(54) INTEGRATED TORQUE JACKET SYSTEMS AND METHODS FOR OCT

(71) Applicant: Corning Incorporated, Corning, NY (US)

(72) Inventors: Venkata Adiseshaiah Bhagavatula, Big Flats, NY (US); Theresa Chang, Painted Post, NY (US); Klaus Hartkorn, Painted Post, NY (US); Mark Alan McDermott, Prattsburgh, NY (US); Stephen Quenton Smith, Lawrenceville, PA (US)

(21) Appl. No.: 14/722,328

(22) Filed: May 27, 2015

Related U.S. Application Data
(60) Provisional application No. 62/007,512, filed on Jun. 4, 2014.

(51) Int. Cl. G02B 6/36; G02B 6/44; A61B 5/00

(52) U.S. Cl. CPC G02B 6/3604; A61B 5/0066; G02B 6/4415; A61B 2562/16

(57) ABSTRACT

Integrated torque jacket systems and methods for optical coherence tomography are disclosed. The system includes an optical fiber cable having an optical fiber surrounded by an outer jacket. An optical probe is operably attached to the distal end of the optical fiber cable. The optical fiber cable includes either a plurality of low-friction bearings or a spiral member operably attached thereto along its length, thereby defining the integrated torque jacket system. The integrated torque jacket system resides within the flexible guide tube with a close fit that allows for rotation and axial translation of the integrated torque jacket system within the guide tube interior. The integrated torque jacket system serves to transfer torque and axial translation applied at its proximal end to the distal end to rotate and axially translate the optical probe within the guide tube.

### CROSS-REFERENCE TO RELATED APPLICATIONS

This application claims the benefit of priority under 35 U.S.C. §119 of U.S. Provisional Application Ser. No. 62/007,512 filed on Jun. 4, 2014 the contents of which are relied upon and incorporated herein by reference in their entirety.

### FIELD

The present disclosure relates to optical coherence tomography (OCT), and in particular relates to an integrated torque jacket systems and methods for use in an OCT.

The entire disclosure of any publication or patent document mentioned herein is incorporated by reference, including U.S. Patent Application Publication No. 2013/0223787 and the article entitled “Optical coherence tomography,” by Huang et al., Science, New Series, Vol. 254, No. 5035 (Nov. 22, 1991), 1178-1181.

### BACKGROUND

Optical coherence tomography (OCT) is used to capture a high-resolution cross-sectional image of scattering biological tissues using fiber-optic interferometry. The core of an OCT system is a Michelson interferometer, wherein a first optical fiber is used as a reference arm and a second optical fiber is used as a sample arm. The sample arm includes the sample to be analyzed as well as an optical probe that includes optical components. An upstream light source provides the imaging light. A photodetector is arranged in the optical path downstream of the sample and reference arms.

Optical interference of light from the sample arm and the reference arm is detected by the photodetector only when the optical path difference between the two arms is within the coherence length of the light from the light source. Depth information from the sample is acquired by axially varying the optical path length of the reference arm and detecting the interference between light from the reference arm and scattered light from the sample arm that originates from within the sample.

A three-dimensional image requires high-speed rotation as well as axial translation of the optical probe. This rotation and axial translation carried out in conventional OCT systems through the use of a metal torque tube that is mechanically connected to the probe at a distal end. The torque tube is threaded through a guide tube, which is referred to in the art as an “inner lumen.” The torque tube is driven to rotate and axially translate at its proximal end by a rotary and axial translation actuator and transmits the rotational and axial translation motion to the optical probe at the distal end.

Conventional torque tubes are made of a stainless steel and have a multi-coil spring assembly, which is a relatively complex design and does not offer very good dimensional control. Further, the torque tube must be feed into the inner lumen over long distances, which is difficult to do because of the flexibility of the spring coil. In addition, there is a large amount of surface area contact that can occur between the torque tube and the inner lumen. This surface area contact represents a source of friction that impacts the rotation and axial translation of the optical probe.

It is therefore desirable to simply the mechanism used to impart rotation to the optical probe so that the OCT system is less expensive and easier to use while also reducing the potential contact area and frictional forces.

### SUMMARY

An aspect of the disclosure is an integrated torque jacket (ITJ) system for use with a guide tube of an optical coherence tomography (OCT) system that utilizes a rotating optical probe. The ITJ system includes: an optical fiber cable of diameter DC. The optical fiber cable has an optical fiber surrounded by a jacket and having a length, a proximal end, and a distal end configured to attach to an optical probe. The ITJ system also has a plurality of low-friction bearings operably disposed on the optical fiber cable along its length. The bearings each have a diameter DB>DC. The bearings are sized so that the optical fiber cable and low-friction bearings can be inserted into and rotate within an interior of the flexible guide tube in a close-fit configuration.

Another aspect of the disclosure is OCT assembly that includes: the bearing-based ITJ system described above, and the guide tube, wherein the guide tube has an inner wall that defines the guide tube interior, and wherein the ITJ system resides within the guide tube interior in the tight-fit configuration.

Another aspect of the disclosure is a method of rotating and axial translating an optical probe in an OCT system. The method includes: operably disposing a plurality of low-friction bearings along a length of an optical fiber cable that has a proximal end and a distal end, wherein the optical probe is operably connected to the optical fiber cable at the distal end; inserting the optical fiber cable and low-friction bearings into an interior of a flexible guide tube in a close-fit configuration; and causing a rotation and axial translation of the optical fiber cable at its proximal end so that the optical fiber cable and low-friction bearings and optical probe rotate and axially translate within the interior of the flexible guide tube.

Another aspect of the disclosure is an ITJ system for use with a guide tube of an optical coherence tomography system that utilizes a rotating optical probe. The ITJ system includes: an optical fiber cable having an optical fiber surrounded by a jacket and having a length, a proximal end and a distal end configured to attached to an optical probe; and a spiral member operably disposed on the optical fiber cable along its length, the spiral member having a diameter sized so that the optical fiber cable and spiral can be inserted into and rotate within an interior of the flexible guide tube in a close-fit configuration.

Another aspect of the disclosure is an OCT assembly that includes the spiral-based ITJ system as described above, and the guide tube, wherein the ITJ system resides within the guide tube interior in the close-fit configuration.

Another aspect of the disclosure is a method of rotating and axially translating an optical probe in an OCT system. The method includes: operably disposing a spiral member along a length of an optical fiber cable that has a proximal end and a distal end, wherein the optical probe is operably connected to the optical fiber cable at the distal end; inserting the optical fiber cable and low-friction bearings into an interior of a flexible guide tube in a close-fit configuration; and causing a rotation and axial translation of the optical fiber cable at its proximal end so that the spiral member and optical probe within the interior of the flexible guide tube.

Additional features and advantages are set forth in the Detailed Description that follows, and in part will be readily apparent to those skilled in the art from the description or recognized by practicing the embodiments as described in the written description and claims hereof, as well as the appended drawings. It is to be understood that both the foregoing general description and the following Detailed Description are merely exemplary, and are intended to provide an overview or framework to understand the nature and character of the claims.

### BRIEF DESCRIPTION OF THE DRAWINGS

The accompanying drawings are included to provide a further understanding, and are incorporated in and constitute a part of this specification. The drawings illustrate one or more embodiment(s), and together with the Detailed Description serve to explain principles and operation of the various embodiments. As such, the disclosure will become more fully understood from the following Detailed Description, taken in conjunction with the accompanying Figures, in which:

FIG. 1 is a cross-sectional view of the probe-end portion of an example prior art OCT system;

FIG. 2A is a partially exploded side view of a first example embodiment of an example OCT system according to the disclosure that includes an example integrated torque jacket (ITJ) system;

FIG. 2B is a close-up side view of an example ITJ system of the OCT system of FIG. 2A, wherein the ITJ system is formed by the optical fiber cable and bearings arranged along the length of the optical fiber cable;

FIG. 2C is a front-elevated partial cut-away view the example ITJ system of FIG. 2B;

FIG. 3A is similar to FIG. 2A and shows the assembled OCT system wherein the ITJ system resides within the interior of a guide tube in a close-fit configuration;

FIG. 3B is similar to FIG. 2B and shows a portion of the ITJ system as disposed within the interior of the guide tube in a close-fit configuration;

FIG. 3C is similar to FIG. 2C and shows a portion of the ITJ system as disposed within the interior of the guide tube in a close-fit configuration;

FIGS. 4A and 4B are close-up side views of example bearings that include grooves formed in outer surface;

FIG. 5 is a close-up cross-sectional view of the optical probe operably arranged within the interior of the guide tube, with the back-end portion of the optical probe having a low-friction coating to facilitate smooth rotation and axial of the probe within the guide tube;

FIG. 6 is a close-up, cross-sectional view of the ITJ system arranged in the interior of the guide tube and illustrating an example wherein the guide tube inner wall includes a low-friction coating to facilitate smooth rotation and axial of ITJ system within the guide tube;

FIG. 7 is similar to FIG. 6 and shows an example wherein the bearing outer surface includes a low-friction coating to facilitate smooth rotation and axial of ITJ system within the guide tube;

FIG. 8A is photograph of TexMatte material with PMMA particles having a size in the range from 25 μm to 30 μm;

FIG. 8B is a plot of the frictional force FF (grams, g) versus distance (relative units) for three different sets of measurements of the measured frictional force for white ink as a control material;

FIG. 8C is the same plot as FIG. 8B but based on data of the measured frictional force FF for the white ink material coated with TexMatte 6025 beads and a 20% F-acrylate low-friction additive, wherein the plot represents an average of three sets of measurements and shows a substantial reduction in the frictional force FF as compared to that of the control material as shown in FIG. 8B; and

FIGS. 9A through 9E are close-up side views of example ITJ systems that employ a spiral member operably disposed on the optical fiber cable, and showing the respective ITJ systems within the interior of a guide tube in a close-fit configuration to form respective OCT assemblies.

### DETAILED DESCRIPTION

Reference is now made in detail to various embodiments of the disclosure, examples of which are illustrated in the accompanying drawings. Whenever possible, the same or like reference numbers and symbols are used throughout the drawings to refer to the same or like parts. The drawings are not necessarily to scale, and one skilled in the art will recognize where the drawings have been simplified to illustrate the key aspects of the disclosure.

The claims as set forth below are incorporated into and constitute part of this Detailed Description.

The entire disclosure of any publication or patent document mentioned herein is incorporated by reference.

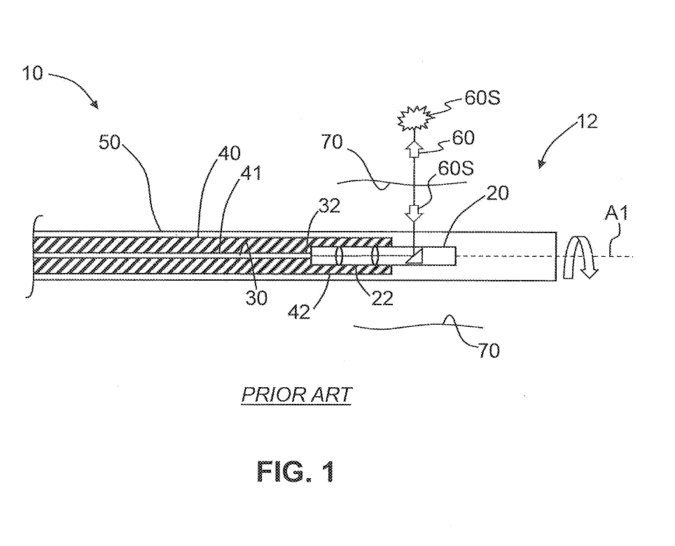

FIG. 1 is a cross-sectional view of an example prior art OCT system 10 showing the probe-end portion 12. The OCT system 10 includes an optical probe (“probe”) 20 that is operably connected to an end 32 of an optical fiber 30. At least a portion of probe 20 is transparent. The optical fiber 30 is supported within a channel 41 of a metal (e.g., stainless steel) torque tube 40. It is noted here that optical fiber 30 and torque tube 40 are separate components that need to be formed separately and then be mechanically combined so that the optical fiber 30 is secured within the channel of the torque tube. The torque tube 40 is a multi-coil spring assembly made of a metal such as stainless steel.

An end portion 22 of probe 20 is attached to an end portion 42 of the torque tube. In an example, the end portion 22 of probe 20 is made of metal, e.g., stainless steel. The torque tube 40 resides within a guide tube (or inner lumen) 50 and is free to rotate and axially translate therein, though there is typically some contact between the torque tube and guide tube, i.e., there is a close-fit between the torque tube and guide tube. Torque tube 40 has a constant diameter and thus represents a configuration that presents a maximum amount of surface area to guide tube 50.

Guide tube 50 is transparent to light 60 at least at probe-end portion 12. In an example, a (transparent) balloon (not shown) is used to create space for the probe-end portion 12 within tissue or vessel 70. The probe-end portion 12 of OCT system 10 is inserted into a catheter or endoscope (not shown) for insertion into the body to be examined.

The light 60 originates from a light source (not shown) and travels down optical fiber 30 to end 32. This light exits end 32 of optical fiber 30 and is directed by probe 20 to the surrounding tissue or vessel 70. Light 60 generates scattered light 60S from the tissue or vessel 70, and some of this scattered light returns to and is captured by probe 20 and directed back to optical fiber end 32. This returned light travels back down optical fiber 30 toward the light source and is then interferometrically processed to generate the OCT image according to methods known in the art.

As noted above, this configuration based on the use of torque tube 40 and guide tube 50 is relatively complicated and experiences frictional forces between the torque tube and the guide tube that adversely impact the operation of OCT system 10.

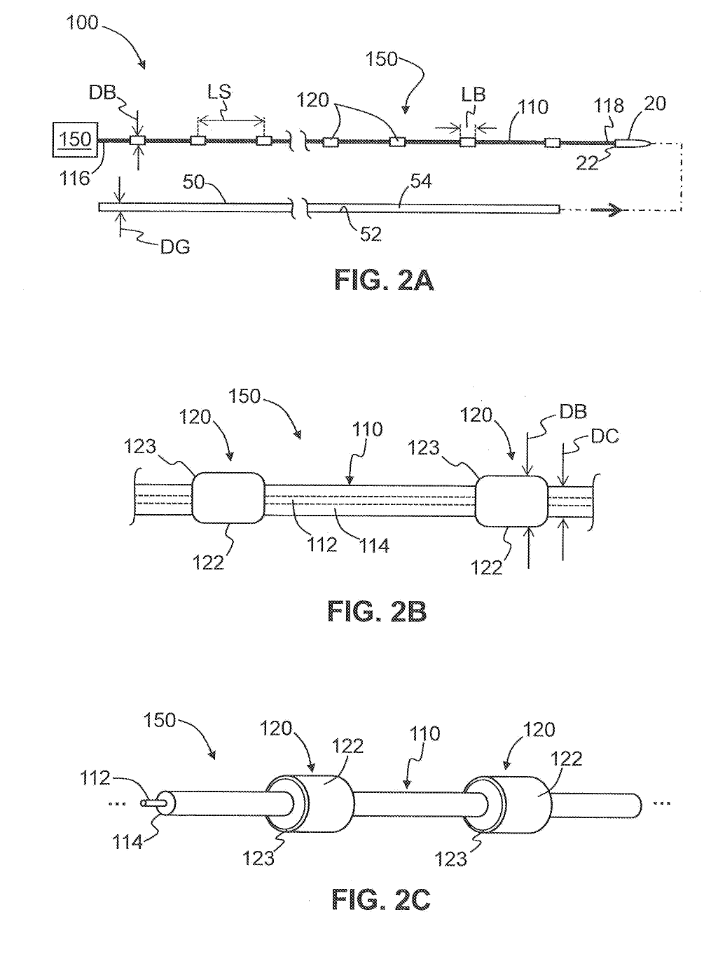

FIG. 2A is a partially exploded side view of a first example embodiment of an example OCT system 100 that includes an example integrated torque jacket (ITJ) system 150 according to the disclosure. FIG. 2B is a close-up side view of a portion of an example ITJ system 150, while FIG. 2C is a front-elevated and partial cut-away view of the ITJ system.

The ITJ system 150 is formed from an optical fiber cable 110. Optical fiber cable 110 includes an optical fiber 112 (shown in phantom in FIG. 2B) and an outer jacket 114 that surrounds the optical fiber. Optical fiber cable 110 can also be referred to as a “jacketed optical fiber.” Optical fiber cable 110 can be formed to have a variety of diameters DC, and in one example 500 μm≦DC≦1000 μm. Example materials for optical fiber jacket 114 include PVC, thermoplastic elastomer (e.g., HYTREL), polyethylene, nylon, polymers, etc. Optical fiber 112 can be a single-mode fiber or a multimode fiber.

The process for forming optical fiber cables 110 are well known in the art. In particular, optical fiber cable 110 is formed using a manufacturing operation that forms the optical fiber 112 and outer jacket 114 as part of the same manufacturing operation, i.e., a drawing operation to form the optical fiber and a coating operation that forms the outer jacket on the optical fiber. In an example, outer jacket 114 is formed from a single dielectric material. Further in an example, outer jacket 114 contains no support elements or other structural elements so that the optical fiber cable 110 is maximally flexible in all directions, i.e., does not have a preferred bending direction. Optical fiber cables 110 are known to be flexible.

In one example, ITJ system 150 includes bearing elements (“bearings”) 120 operably disposed along the length of optical fiber cable 110. Bearings 120 have a low-friction outer surface 122. The bearings 120 have a diameter DB and an axial length LB. In an example, the bearing diameter DB is in the range 700 microns≦DB≦1300 microns, with the condition that DB>DC.

Adjacent bearings 120 are shown as having a center-to-center axial spacing LS. In one example, the spacing LS is uniform (i.e., bearings 120 have a constant pitch), while in another example the spacing LS can vary along the length of optical fiber cable 110 (i.e., bearings 120 can have a variable pitch). Likewise, the axial length LB of bearings 120 can all be the same or can vary between some or all of the bearings.

In an example, bearings 120 are fixed, secured, attached, etc. to optical fiber cable 110 using conventional means. In one example, bearings 120 are fixed to optical fiber cable 110 using an adhesive, while in another example the bearings are crimped to the optical fiber cable, while in yet another example are thermally attached (e.g., via heat shrinking). In an example, bearings 120 have rounded or chamfered edges 123 (see FIGS. 2B and 2C) that reduce friction. In an example, bearings 120 are in the form of low-friction beads, which in examples are ovoidal, spheroidal or spherical.

With reference again to FIG. 2A, optical fiber cable 110 includes a proximal end 116 that is operably connected to a rotary and axial translation actuator (“actuator”) 150 and a distal end 118 that is operably connected to back-end portion 22 of probe 20.

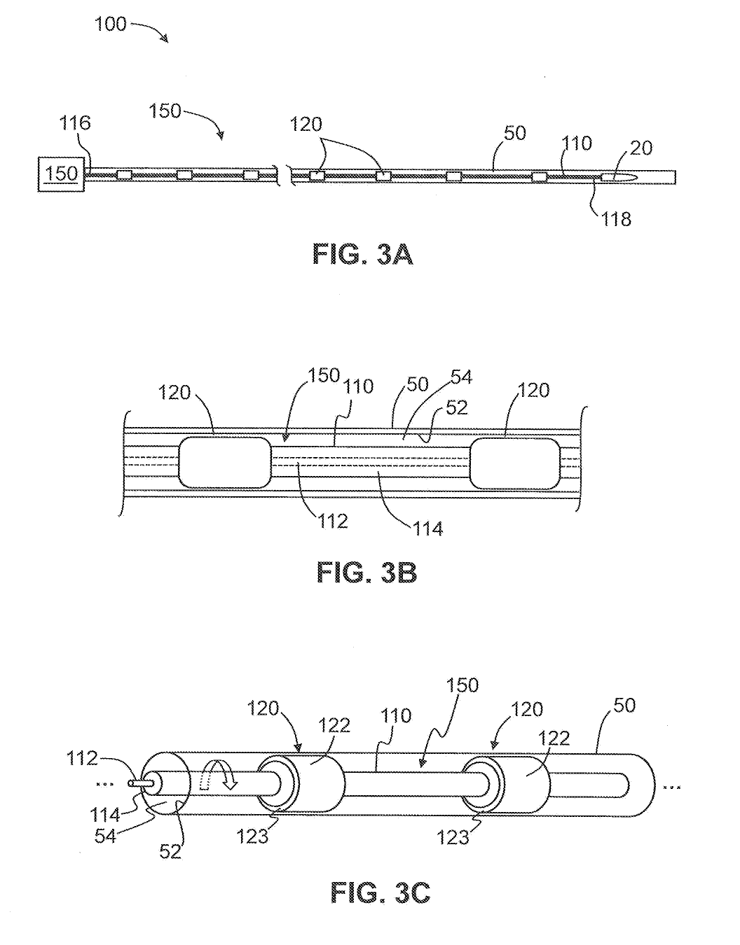

FIG. 3A is similar to FIG. 2A and shows the assembled OCT system 100, while FIGS. 3B and 3C are similar to FIGS. 2B and 2C but for an assembled portion of the OCT system showing the ITJ system 150 operably disposed within guide tube 50. The guide tube 50 has an inner wall 52 that defines an interior 54 with a generally circular cross-section having an inner diameter DG. The diameter DB of bearings 120 is slightly smaller than diameter DG so that ITJ system 150 has a close fit within guide tube interior 54, which in an example is made of a flexible transparent polymer. The combination of the guide tube 50 and ITJ system 150 operably disposed within the guide tube interior 54 defines an OCT assembly.

The clearance C=(DG−DB) of bearings 120 within guide tube 50 is selected as a balance between preventing uncontrolled lateral movement (“lashing”) of the bearings during rotation and axial translation and of ITJ system 150 within guide tube interior 54 with low-friction between bearings 120 and inner wall 152 of the guide tube, including when the guide tube is bent or flexed during the OCT procedure. Thus, the bearings 120 and the interior 54 of guide tube 50 define a close fit, i.e., one in which there is sufficient space for the bearings to rotate within guide tube interior 54 but insufficient space for the bearings to be laterally displaced to a substantial extent, e.g., no more than a few percent of the bearing diameter DB. Thus, in the close fit configuration, the outer surface 122 of bearings 120 loosely contact inner wall 52 of guide tube 50. It is also noted that the amount of area presented by bearings 120 to inner wall 52 of guide tube 50 is substantially less than for a prior art torque tube 40 discussed above that has a constant diameter.

Random manufacturing variations in guide tube 50 and ITJ system 150 can cause an increase in the frictional forces or an increase in lashing of the ITJ system within the guide tube. These variations can lead to non-uniform rotation of probe 20 and can put stress on the various components. This stress can lead to a system failure, e.g., probe 20 becoming disconnected from ITJ system 150. Thus, in an example, the clearance C=(DG−DB) is in the range from 100 μm to 150 μm to define the close-fit configuration and reduce or minimize the adverse effects of the random manufacturing variations.

In an example, guide tube 50 can be formed from polymer using an extrusion or a drawing process. The extrusion processes provides good dimensional control, thereby reducing the potential adverse effects of the aforementioned random manufacturing errors.

The pitch of bearings 120 can be selected to provide minimum contact area between bearing outer surfaces 122 and the inner wall 52 of guide tube 50 while also optimizing torsional rigidity without comprising flexibility.

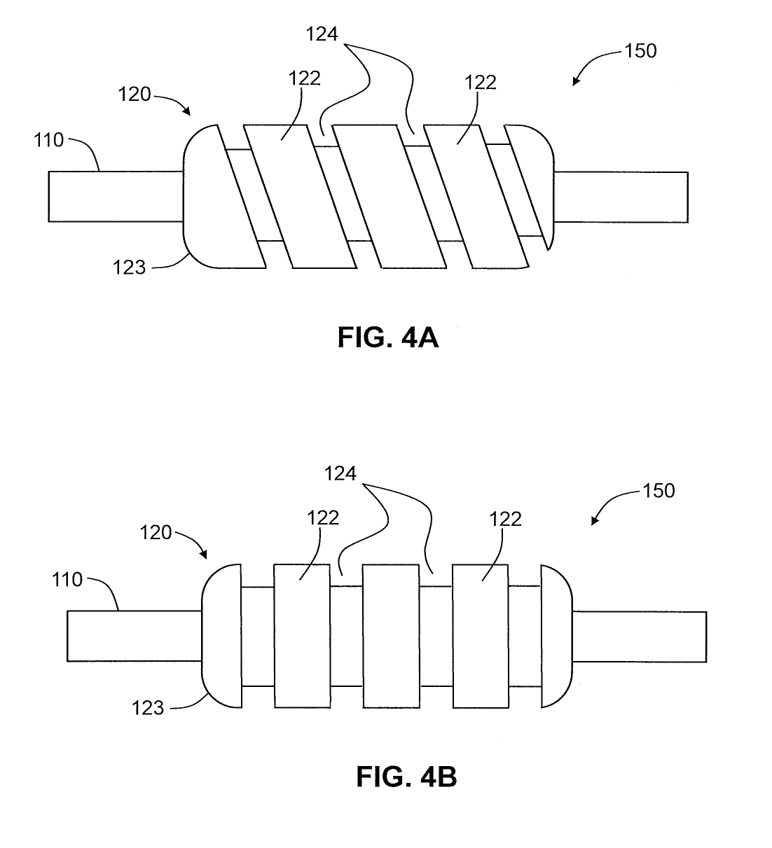

FIGS. 4A and 4B are close-up side views of example bearings 120 that include grooves 124 formed in outer surface 122. Grooves 124 served to further reduce the amount of contact area between outer surface 122 of bearings 120 and the inner wall 52 of guide tube 50.

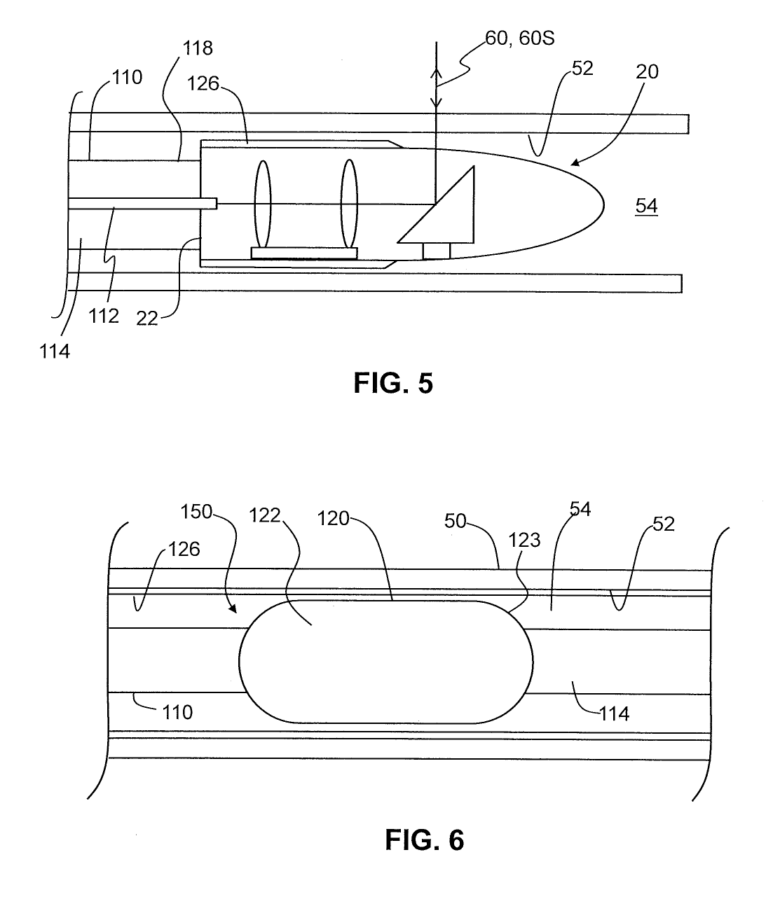

In an example embodiment, one or more components of OCT system 100 can include a low-friction coating. For example, FIG. 5 is a close-up cross-sectional view of probe 20 within guide tube 50. In an example, at least a portion of probe 20 includes a low-friction coating 126 disposed to facilitate the low-friction rotation and axial translation of the probe within interior 54 of guide tube 50.

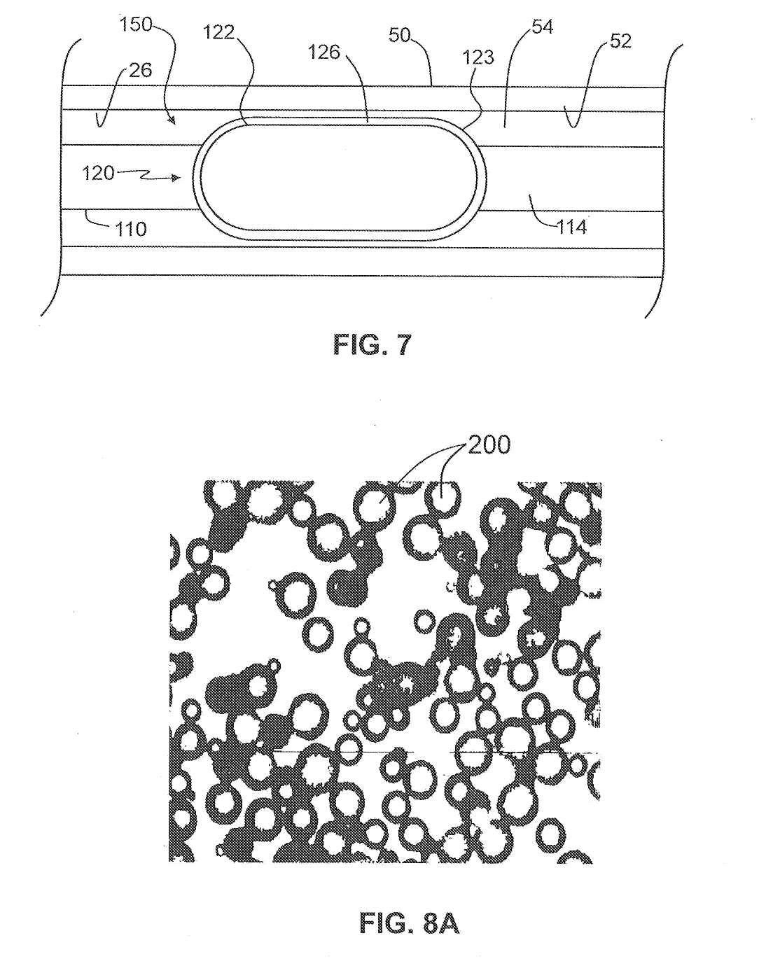

FIG. 6 is a cross-sectional view of a portion of OCT system 100 and illustrates an example embodiment where at least a portion of inner wall 52 includes low-friction coating 126. FIG. 7 is similar to FIG. 6 and shows an example wherein outer surfaces 122 of bearings 120 have a relatively low coefficient of friction (i.e., are low-friction surfaces), while inner wall 154 of guide tube 50 is smooth. In another example, the low-friction outer surfaces 122 of bearings 120 are due to the bearings being made of a low-friction material.

Example low-friction materials include polytetrafluorotethylene, polyimide, polyamide, polyethylene, polysilicone, fluorosilane, fluoroether silanes, silicones, etc. In an example, bearing outer surface 122 (or the low-friction coating 126 thereon) has a coefficient of static friction μₛ<0.5, while in another example, μs<0.1, while yet in another example, μₛ<0.05.

Low-friction coating 126 can be made from any of the known low-friction materials and can be spray coated, spin-coated, dipped, etc. In one example, a TEFLON-based low-friction coating 126 was prepared using 1% TEFLON AF in a fluoroether solvent FC-40 and combined with a solution of adhesion binder (1 Wt % in HFE7200) to produce a solution that was 1 wt % total in polymer mass. The solution was filtered through a coarse paper filter prior to use. An example of using an adhesion binder and the preparation details for non-stick coating materials are described in U.S. Patent Application Publication No. 2012/0189843.

In an example, low-friction coating 126 is applied to metal (e.g., stainless steel) components of OCT system 100. In one example, this can be accomplished by first removing any organic contaminants from the metal surface. Such cleaning can be performed by using an ethanol-soaked wipe and then allowing the surface to dry. In an example, low-friction coating 126 can be applied (e.g., immersion or spraying or dipping) and then cured in an oven by ramping the temperature from 100° C. to 165° C. at 5° C./minute, holding at 165° C. for 15 minutes, and then ramping to 280° C. at 5°/minute, and then holding at 280° C. for 60 minutes.

In another example, low-friction coating can be made from heptadecafluoro-tetrahydrodecyl-trichlorosilane (C₁₀H₄F₁₇Si Cl₃) by combining perfluorosilane with anhydrous heptane. The metal surfaces can then be cleaned and then immersed in the coating solution for 1 minute. Upon removal, the coated metal surfaces can be rinsed with heptane and then ethanol.

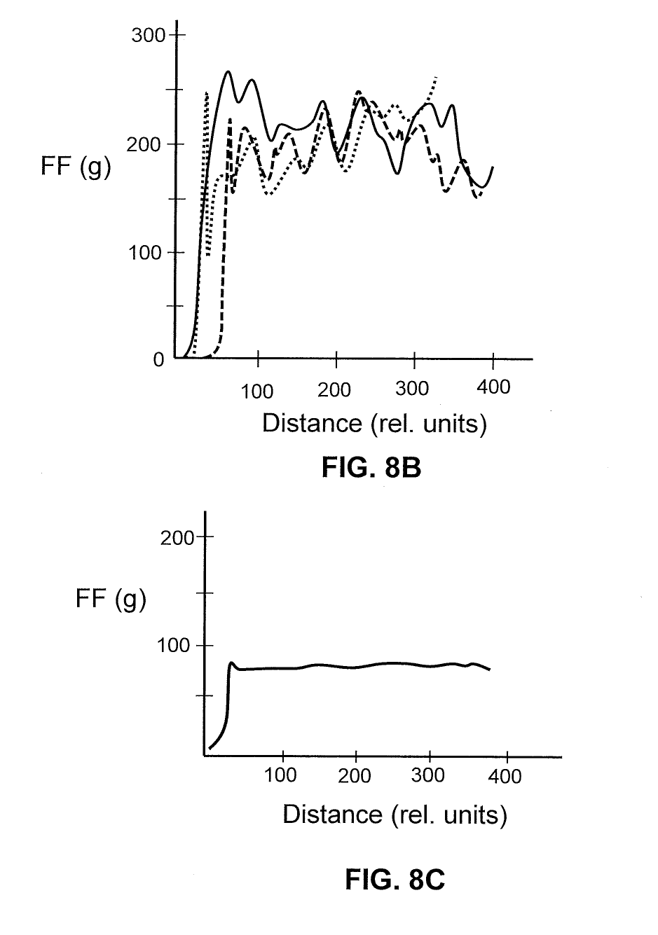

In an example, low-friction coating 126 includes one or more low-friction enhancements, such as low-friction particles and/or additives. The particles and/or additives can also be added to inner surface 52 of guide tube 50 and/or jacket 114 of optical fiber cable 110 during their fabrication. FIG. 8A is photograph of TexMatte material 6025 having PMMA particles 200 having a size in the range from 25 μm to 30 μm.

FIG. 8B is a plot of the friction force FF (grams, g) versus distance (relative units) for white ink as a control material. The plot is based on three sets of measurements obtained using a conventional frictional force measurement device.

FIG. 8C is the same plot as FIG. 8B but for the white ink material coated with a low-friction coating 126 of TexMatte 6025 beads with 20% F-acrylate low-friction additive. The plot of FIG. 8C is based on an average of three sets of measurements and shows that the addition of the beads and the low-friction additive substantially reduces the coefficient of friction of the control material.

An aspect of the disclosure is a method of rotating and axially translating optical probe 20 in OCT system 100. The method includes operably disposing a plurality of the low-friction bearings 120 along the length of optical fiber cable 110, with optical probe 20 being operably connected to the optical fiber cable at the distal end 118. This forms the ITJ system 150 as discussed above. The ITJ system 150 is then inserted into interior 54 of flexible guide tube 50 in a close-fitting configuration. The method further includes causing a rotation and an axial translation of ITJ system 150 at its proximal end, e.g., by activating actuator 150 operably connected thereto. This causes rotation and axial translation of the optical fiber cable 110, the low-friction bearings 120 thereon and the optical probe 20 attached thereto within the interior 54 of the flexible guide tube. Thus, ITJ system 150 transfers the torque and axial translation generated by actuator 150 at the proximal end of optical fiber cable 110 to its distal end, thereby causing the rotation and axial translation of the optical probe. In examples of the method, one or more low-friction coatings 126 are employed on at least one of: the inner wall 52 of guide tube 50; bearings 120; and at least an end portion 22 of optical probe 20.

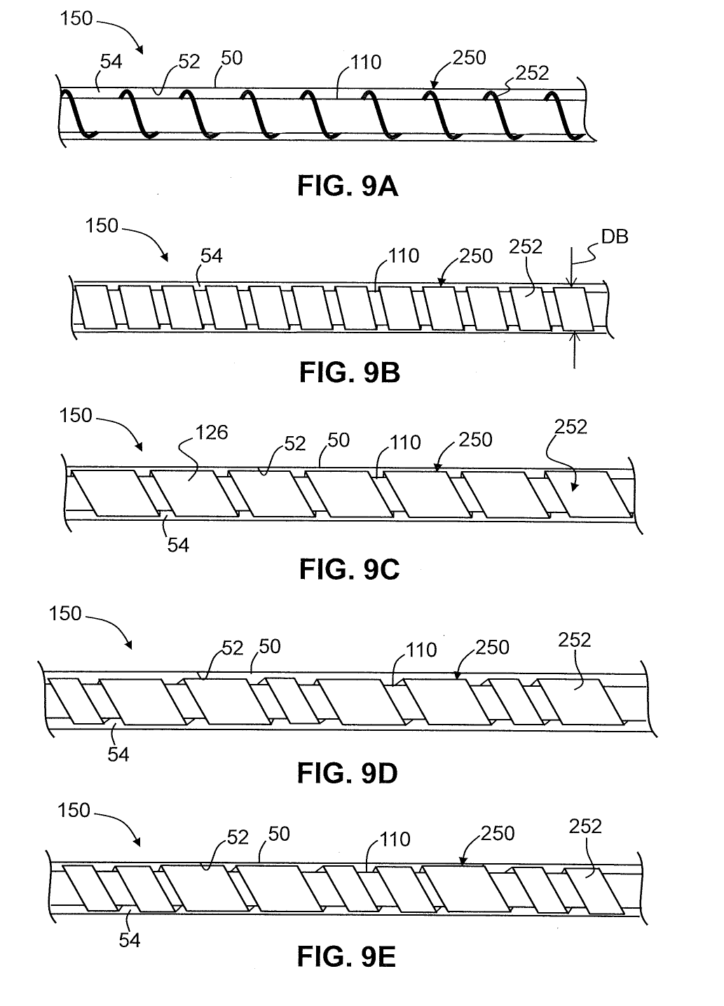

FIGS. 9A through 9E are a close-up side views of different examples of an ITJ system 150 operably disposed in interior 54 of guide tube 50 to form respective OCT assemblies. The ITJ systems of FIGS. 9A through 9E include a spiral member 250 operably disposed on outer jacket 114 of optical fiber cable 110.

In FIG. 9A, spiral member 250 is shown as a coil that is wound around optical fiber cable 110. In examples, the coil can be made of a metal (e.g., copper) or a polymer and can be attached to optical fiber cable 110 using known means, which include using an adhesive or a wax. Though the coil of FIG. 9A is shown as being evenly wound (i.e., having an even pitch), in other examples the coil can be wound to have a variable pitch. Spiral member 250 has an outer surface 252. Spiral member 250 can also be a metal that is coated with a non-metal layer, e.g., a polymer, thermoplastic or wax layer.

FIGS. 9B through 9E show examples of ITJ system 150 where spiral member 250 is formed from tubing material, such as from a polymer tube, and attached to optical fiber cable 110. FIGS. 9B and 9C show two examples of an “even” spiral (i.e., constant pitch), whiles FIGS. 9D and 9E show examples with an uneven spiral (i.e., non-constant pitch). In an example, the polymer tube can be made of a heat-shrink material so that it can be attached to optical fiber cable 110 by the application of heat, such as from a heat gun. In an example, outer jacket 114 can be made extra thick and then have a spiral groove formed therein to define spiral member 250.

FIG. 9B shows the spiral member diameter DS, which is analogous to the bearing diameter DB. Like the case for the bearing-based ITJ system 150, the spiral-based ITJ system has a clearance within guide tube 50 of C=(DG−DS) that in an example is in the range from 100 μm to 150 μm. In an example, 700 microns≦DS≦1300 microns, with the condition that DS>DC. The use of spiral member 250 serves to substantially reduce the amount of surface area presented to inner surface 152 of guide tube 150 as compared to the use of a conventional torque tube 40.

In an examples, spiral member 250 includes a low-friction coating 126, as illustrated in the example ITJ system 150 of FIG. 9C. In another example, spiral member 250 is made of or otherwise includes one or more low-friction materials, such as discussed above in connection with bearings 120.

Another aspect of the disclosure is a method of rotating and axially translating optical probe 20 in OCT system 100 using the spiral-based ITJ system 150. The method includes operably disposing spiral member 250 along the length of optical fiber cable 110, with optical probe 20 being operably connected to the optical fiber cable at the distal end 118. This forms the spiral-based ITJ system 150 as discussed above. The ITJ system 150 is then inserted into interior 54 of flexible guide tube 50 in a close-fitting configuration, thereby defining an OCT assembly. The method further includes causing a rotation and axial translation of ITJ system 150 at its proximal end, e.g., by activating actuator 150 operably connected thereto. This causes rotation and axial translation of the optical fiber cable 110, the spiral member 250 thereon and the optical probe 20 attached thereto within the interior 54 of the flexible guide tube. Thus, ITJ system 150 transfers the torque and axial translation generated by rotary and axial translation actuator 150 at the proximal end of optical fiber cable 110 to its distal end, thereby causing the rotation and axial translation of the optical probe. In examples of the method, one or more low-friction coatings 126 are employed on at least one of: the inner wall 52 of guide tube 50; spiral member 250; and at least an end portion 22 of optical probe 20.

It will be apparent to those skilled in the art that various modifications to the preferred embodiments of the disclosure as described herein can be made without departing from the spirit or scope of the disclosure as defined in the appended claims. Thus, the disclosure covers the modifications and variations provided they come within the scope of the appended claims and the equivalents thereto.

What is claimed is:

1. An integrated torque jacket system for use with a guide tube of an optical coherence tomography system that utilizes a rotating optical probe, comprising:
an optical fiber cable having an optical fiber surrounded by a jacket and having a length, a proximal end, and a distal end configured to attach to an optical probe, the optical fiber cable having a diameter DC; and
a plurality of low-friction bearings operably disposed on the optical fiber cable along its length, the bearings each having a diameter DB>DC and sized so that the optical fiber cable and low-friction bearings can be inserted into and rotate within an interior of the flexible guide tube in a close-fit configuration.

2. The integrated torque jacket system according to claim 1, wherein each of the low-friction bearings have a low-friction outer surface defined by a low-friction coating.

3. The integrated torque jacket system according to claim 1, wherein the low-friction bearings have a static coefficient of friction μₛ≦0.1.

4. The integrated torque jacket according to claim 1, wherein the low-friction bearings have an outer surface that includes one or more slots formed therein.

5. The integrated torque jacket system according to claim 1, wherein the plurality of bearings have a constant pitch.

6. The integrated torque jacket system according to claim 1, wherein each of the bearings has the same axial length.

7. The integrated torque jacket system according to claim 1, wherein the optical fiber cable comprises a tight-buffered optical fiber cable.

8. An optical coherence tomography (OCT) assembly, comprising:
the integrated torque jacket system of claim 1; and
the guide tube, wherein the guide tube has an inner wall that defines the guide tube interior, and wherein the integrated torque jacket system resides within the guide tube interior in the tight-fit configuration.

9. The OCT assembly according to claim 8, wherein the interior of the guide tube has a diameter DG, and wherein the bearings and guide tube define a clearance of C=(DG−DB) in the range from 100 μm to 150 μm.

10. The OCT assembly according to claim 8, further comprising the optical probe operably connected to the distal end of the optical fiber cable.

11. The OCT assembly according to claim 10, further including at least one low-friction coating applied to at least one of: one or more of the bearings, the inner wall of the guide tube, and at least a portion of the optical probe.

12. The OCT assembly according to claim 10, wherein the low-friction coating includes a material selected from the group of materials comprising: polytetrafluorotethylenes, TEFLON AF, polyimides, polyamides, polyethylenes, polysilicones, fluorosilanes, fluoroether silanes, and silicones.

13. A method of rotating and axial translating an optical probe in an optical coherence tomography (OCT) system, comprising:
operably disposing a plurality of low-friction bearings along a length of an optical fiber cable that has a proximal end and a distal end, wherein the optical probe is operably connected to the optical fiber cable at the distal end;
inserting the optical fiber cable and low-friction bearings into an interior of a flexible guide tube in a close-fit configuration; and
causing a rotation and an axial translation of the optical fiber cable at its proximal end so that the optical fiber cable and low-friction bearings and optical probe rotate and axially translate within the interior of the flexible guide tube.

14. The method according to claim 13, wherein the close-fit is defined by a clearance between each of the bearings and an inner wall of the flexible guide tube of between 100 μm and 150 μm.

15. The method according to claim 13, wherein the causing of the rotation and translation of the optical fiber cable at its proximal end includes operably connecting the proximal end of the fiber cable to a rotary and axial translation actuator and activating the rotary and axial translation actuator.

16. The method according to claim 13, wherein each of the plurality of bearings includes a low-friction outer surface having a coefficient of static friction μₛ≦0.1.

17. The method according to claim 13, wherein the optical fiber cable has a diameter in the range from 500 μm to 1000 μm.

18. The method according to claim 17, wherein each of the plurality of bearings has a diameter DB in the range from 700 microns to 1300 microns.

19. The method according to claim 13, including providing at least one of the guide tube interior, the plurality of bearings and the optical probe with at least one low-friction coating.

20. The method according to claim 19, wherein the at least one low-friction coating includes at least one of a low-friction additive and low-friction beads.

21. An integrated torque jacket system for use with a guide tube of an optical coherence tomography system that utilizes a rotating optical probe, comprising:
an optical fiber cable having an optical fiber surrounded by a jacket and having a length, a proximal end and a distal end configured to attached to an optical probe; and
a spiral member operably disposed on the optical fiber cable along its length, the spiral member having a diameter sized so that the optical fiber cable and spiral can be inserted into and rotate within an interior of the flexible guide tube in a close-fit configuration.

22. The integrated torque jacket system according to claim 21, wherein the spiral member comprises at least one of a metal, a polymer and a thermoplastic.

23. The integrated torque jacket system according to claim 21, wherein the spiral member is evenly wound about the optical fiber cable to define an even pitch.

24. The integrated torque jacket system according to claim 21, wherein the spiral member comprises a low-friction coating having a coefficient of static friction μₛ≦0.1.

25. The integrated torque jacket system according to claim 21, wherein the spiral member is made of a low-friction material.

26. An optical coherence tomography (OCT) assembly, comprising:
the integrated torque jacket system of claim 21; and
the guide tube, wherein the integrated torque jacket system resides within the guide tube interior in the close-fit configuration.

27. The OCT assembly according to claim 26, wherein the interior of the guide tube has a diameter DG, the spiral member has a diameter DS, and wherein the spiral member and guide tube define a clearance of C=(DG−DS) in the range from 100 μm to 150 μm.

28. The OCT assembly according to claim 26, further comprising the optical probe operably connected to the distal end of the optical fiber cable.

29. The OCT assembly according to claim 28, further including at least one low-friction coating applied to at least one of: the spiral member, an inner wall of the guide tube, and at least a portion of the optical probe.

30. A method of rotating and axially translating an optical probe in an optical coherence tomography (OCT) system, comprising:
operably disposing a spiral member along a length of an optical fiber cable that has a proximal end and a distal end, wherein the optical probe is operably connected to the optical fiber cable at the distal end;
inserting the optical fiber cable and low-friction bearings into an interior of a flexible guide tube in a close-fit configuration; and
causing a rotation and axial translation of the optical fiber cable at its proximal end so that the optical fiber cable, the spiral member and optical probe rotate and axially translate within the interior of the flexible guide tube.

31. The method according to claim 30, wherein the close-fit is defined by a clearance between the spiral member and an inner wall of the flexible guide tube of between 100 μm and 150 μm.

32. The method according to claim 30, wherein the causing of the rotation includes operably connecting the proximal end of the fiber cable to a rotary and axial translation actuator and activating the rotary and axial translation actuator.
